(19) United States
(12) Patent Application Publication
(10) Pub. No.: US 2025/0099084 A1
(43) Pub. Date: Mar. 27, 2025

(54) ULTRASONIC MEASUREMENT APPARATUS

(71) Applicant: SEIKO EPSON CORPORATION, TOKYO (JP)

(72) Inventors: Mio SASAKI, Shiojiri-shi (JP); Seiji IZUO, Shiojiri-shi (JP)

(21) Appl. No.: 18/898,749

(22) Filed: Sep. 27, 2024

(30) Foreign Application Priority Data
Sep. 27, 2023 (JP) 2023-164714

(51) Int. Cl. A61B 8/00

(52) U.S. Cl. CPC A61B 8/54; A61B 8/4444; A61B 8/461

(57) ABSTRACT

An ultrasonic measurement apparatus according to the present disclosure is an ultrasonic measurement apparatus configured to transmit an ultrasonic wave to a measurement target and receive the ultrasonic wave reflected inside the measurement target, and includes an ultrasonic sensor configured to transmit and receive the ultrasonic wave and output an ultrasonic signal by receiving the ultrasonic wave, an processor configured to output a measurement start request for instructing start of the ultrasonic measurement, and a processor configured to record the ultrasonic signal in a storage, wherein the processor records, in the storage, the ultrasonic signal after a predetermined time elapses from the output of the measurement start request.

The present application is based on, and claims priority from JP Application Serial Number 2023-164714, filed Sep. 27, 2023, the disclosure of which is hereby incorporated by reference herein in its entirety.

### BACKGROUND

### 1. Technical Field

The present disclosure relates to an ultrasonic measurement apparatus.

### 2. Related Art

For ultrasonic measurement in which an ultrasonic wave is transmitted to a measurement target, and then the ultrasonic wave reflected inside the measurement target is received, it is necessary to place an acoustic matching material (such as gel), which has an acoustic impedance between that of an ultrasonic sensor and that of the measurement target, between the ultrasonic sensor and the measurement target.

For example, an apparatus described in JP-A-2023-83445 is an ultrasonic diagnostic apparatus in which an ultrasonic probe and a diagnostic apparatus main body are connected to each other via wireless communication, and the ultrasonic probe is provided with an oscillator array and an acoustic lens. Further, when the ultrasonic probe performs measurement of a subject (measurement target), an ultrasonic gel is applied onto the acoustic lens, and the ultrasonic probe is brought into contact with the subject (measurement target).

The ultrasonic diagnostic apparatus described above requires an application step of applying the acoustic matching material such as an ultrasonic gel between the ultrasonic sensor (the oscillator array and the acoustic lens) and the measurement target and an operation step of instructing the start of ultrasonic measurement. In general, a diagnostic method using the ultrasonic probe differs for each user. For example, a user may bring the ultrasonic probe into contact with the measurement target after the application step, execute the operation step, and then start the measurement (first diagnostic procedure). Further, another user may execute the application step after the operation step, and then bring the ultrasonic probe into contact with the measurement target (second diagnostic procedure).

Herein, in a case of the first diagnostic procedure, an ultrasonic signal based on an ultrasonic wave reflected inside the measurement target can be obtained directly after the ultrasonic measurement is stared. However, in the second diagnostic procedure, an ultrasonic wave reflected inside the measurement target cannot be received between a time at which the ultrasonic measurement is started and a time at which the ultrasonic probe is brought into contact with the measurement target, and hence an appropriate ultrasonic signal cannot be obtained.

In particular, in the second diagnostic procedure, when the ultrasonic measurement is performed successively for a predetermined time, and a measurement result is output based on an average of the ultrasonic signals obtained from the measurement, there is a period in which a correct ultrasonic signal cannot be obtained, and hence the measurement accuracy may be degraded.

### SUMMARY

According to a first aspect of the present disclosure, an ultrasonic measurement apparatus is an ultrasonic measurement apparatus configured to transmit an ultrasonic wave to a measurement target and receive the ultrasonic wave reflected inside the measurement target, and includes an ultrasonic sensor configured to transmit and receive the ultrasonic wave and output an ultrasonic signal by receiving the ultrasonic wave, an processor configured to output a measurement start request for instructing start of the ultrasonic measurement, and a processor configured to record the ultrasonic signal in a storage, wherein the processor records, in the storage, the ultrasonic signal after a predetermined time elapses from the output of the measurement start request.

### BRIEF DESCRIPTION OF THE DRAWINGS

FIG. 1 is a block diagram illustrating an overview configuration of an ultrasonic measurement apparatus of a first embodiment.

FIG. 2 is a flowchart illustrating an ultrasonic measurement method in the first embodiment.

FIG. 3 is a flowchart illustrating the ultrasonic measurement method in the first embodiment.

FIG. 4 is a diagram illustrating one example of a top screen displayed by a display unit of an external device in the first embodiment.

FIG. 5 is a diagram illustrating one example of a stand-by time screen displayed by the display unit of the external device in the first embodiment.

FIG. 6 is a block diagram illustrating an overview configuration of an ultrasonic measurement apparatus of a second embodiment.

FIG. 7 is a flowchart illustrating an ultrasonic measurement method in the second embodiment.

FIG. 8 is a block diagram illustrating an overview configuration of an ultrasonic measurement apparatus of a third embodiment.

FIG. 9 is a diagram illustrating one example of a B-mode image generated based on an ultrasonic signal when an acoustic matching material is not applied onto a transmission/reception surface of an ultrasonic sensor.

FIG. 10 is a diagram illustrating one example of the B-mode image generated based on an ultrasonic signal when the acoustic matching material is applied onto the transmission/reception surface of the ultrasonic sensor.

FIG. 11 is a flowchart illustrating an ultrasonic measurement method in the third embodiment.

### DESCRIPTION OF EMBODIMENTS

### First Embodiment

An ultrasonic measurement apparatus of a first embodiment is described below.

In the present embodiment, it is assumed that a measurement target is a human body (living body), and description is made on an apparatus that measures a state inside the living body by using an ultrasonic wave while an ultrasonic probe including an ultrasonic sensor is fixed with respect to the living body. For example, an ultrasonic wave is transmitted into the living body, and the ultrasonic wave reflected from a tissue (for example, a muscle, fat, an organ, and a blood vessel) is received. With this, the measurement of the thickness of each tissue, the measurement of a pulse rate based on a change in blood vessel position, and the like can be performed.

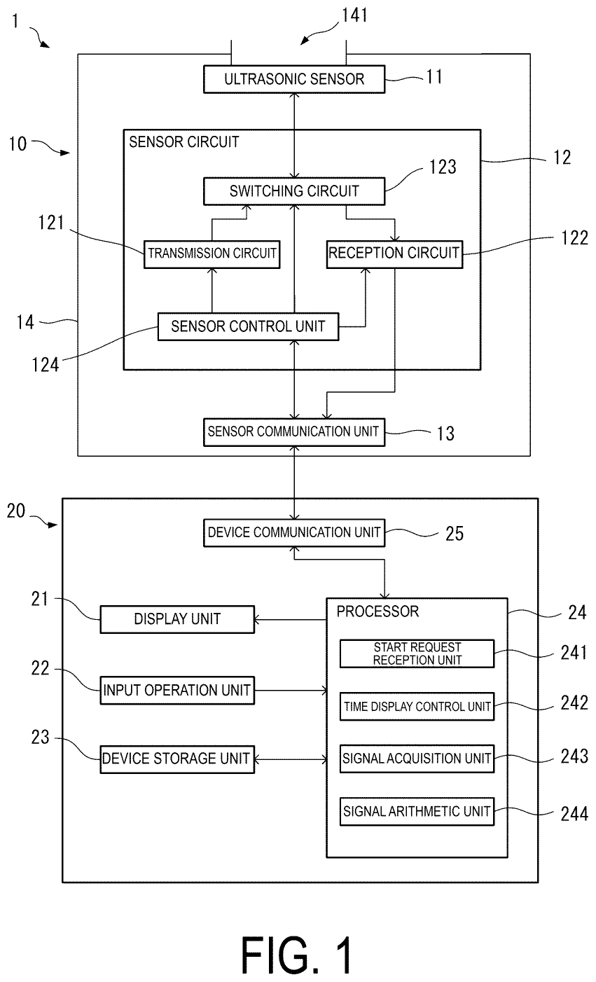

FIG. 1 is a block diagram illustrating an overview configuration of the ultrasonic measurement apparatus 1 of the present embodiment.

As illustrated in FIG. 1, the ultrasonic measurement apparatus 1 of the present embodiment is configured to include an ultrasonic probe 10 and an external device 20.

### Configuration of Ultrasonic Probe 10

The ultrasonic probe 10 is configured to be mountable on a living body, and performs the ultrasonic measurement with respect to an inside of a human body.

The ultrasonic probe 10 includes an ultrasonic sensor 11, a sensor circuit unit 12, a sensor communication unit 13, and a housing 14. Further, although omitted in illustration, a battery is provided in the housing 14 of the ultrasonic probe 10, and the power is supplied from the battery to the sensor circuit unit 12 and the sensor communication unit 13. Note that, in the present embodiment, the power is supplied from the battery to the ultrasonic sensor 11 via the sensor circuit unit 12.

The ultrasonic sensor 11 transmits an ultrasonic wave to a living body, and receives the ultrasonic wave reflected inside the living body. The ultrasonic sensor 11 is not particularly limited as long as it is configured to transmit and receive the ultrasonic wave. For example, a bulk-type ultrasonic element may be used. The bulk-type ultrasonic element transmits an ultrasonic wave by oscillating a piezoelectric body itself by applying a voltage to the piezoelectric body, and detects the reflected wave from an ultrasonic signal output by deformation of the piezoelectric body itself due to the reflected wave. Alternatively, a thin-type ultrasonic element may be used. In the thin-type ultrasonic element, a plurality of ultrasonic transducers each of which includes a piezoelectric element arranged in a thin-layer oscillation part are arranged in an array, and each of the oscillation parts is oscillated by application of the voltage to the piezoelectric element. With this, the ultrasonic wave is transmitted. In such a thin-type ultrasonic element, the ultrasonic signal is output from the piezoelectric element by oscillating an oscillation film by the reflected wave.

When the ultrasonic sensor 11 is arranged in a small range, the thin-type ultrasonic element is suitably used in order to achieve thickness reduction and size reduction. Further, for example, among the ultrasonic transducers arranged in an array including N rows and M columns, one or a plurality of ultrasonic transducers arrayed in the row direction are driven as one channel. With this, a plurality of independent channels can be arranged in parallel in the column direction. In this case, the delay timing of each of the channels is controlled independently, and hence the transmission direction of the ultrasonic wave can be swept along the column direction, or the ultrasonic wave can be converged at a freely-selected depth.

Further, the plurality of ultrasonic transducers can be driven independently from each other. In such a case, the transmission direction of the ultrasonic wave can be swept along the column direction and the row direction, and the ultrasonic wave can be converged at a freely-selected position.

Note that, similarly in a case of the bulk-type ultrasonic element, the transmission direction of the ultrasonic wave can be swept along a freely-selected direction, and the ultrasonic wave can be converged at a freely-selected depth, by arranging a plurality of bulk-type ultrasonic elements in one direction. However, in the bulk-type ultrasonic element, the size of one element is larger than one ultrasonic transducer forming the thin-type ultrasonic element. Therefore, in general, the ultrasonic wave is converged at a predetermined position by using an acoustic lens.

Further, when the reflected wave from the living body is received, the ultrasonic sensor 11 outputs an ultrasonic signal having a signal value corresponding to a sound pressure of the received ultrasonic wave. When the ultrasonic sensor 11 includes a plurality of channels independent from each other, the ultrasonic signal from each of the channels is output independently.

In the present embodiment, when an instruction for staring the measurement (measurement start request) is input, the ultrasonic sensor 11 starts the ultrasonic measurement, and then the ultrasonic signal is output. Note that, although details thereof are described later, the ultrasonic signal is actually taken in as data when a predetermined time being set in advance elapses after the input of the measurement start request.

The sensor circuit unit 12 includes a transmission circuit 121, a reception circuit 122, a switching circuit 123, and a sensor control unit 124.

The transmission circuit 121 generates a driving voltage for transmitting the ultrasonic wave from the ultrasonic sensor 11, and outputs the driving voltage to the ultrasonic sensor 11 via the switching circuit 123.

The reception circuit 122 executes noise removal processing, signal amplification processing, and A/D conversion processing for the ultrasonic signal that is input from the ultrasonic sensor 11 via the switching circuit 123. With this, the ultrasonic signal is converted into digital data. In the present embodiment, the ultrasonic signal converted into the digital data is transmitted directly from the sensor communication unit 13 to the external device 20.

The switching circuit 123 execute switching between transmission connection for connecting the ultrasonic sensor 11 and the transmission circuit 121 to each other and reception connection for connecting the ultrasonic sensor 11 and the reception circuit 122 to each other.

The sensor control unit 124 controls driving of the ultrasonic sensor 11, based on the measurement start request that is input from the external device 20, and executes ultrasonic measurement processing. Specifically, the sensor control unit 124 switches the connection state of the switching circuit 123 between the transmission connection and the reception connection in an alternating manner at a predetermined cycle. With this, the ultrasonic signal changing over time can be received. Further, the sensor control unit 124 transmits the ultrasonic signal subjected to the signal processing by the reception circuit 122, to the external device 20 via the sensor communication unit 13.

The sensor communication unit 13 communicably connects the ultrasonic probe 10 and the external device 20 to each other. In the present embodiment, the sensor communication unit 13 is connected to the external device 20 via wireless communication. Wireless communication is not particularly limited, and examples thereof may include infrared communication, Bluetooth (registered trademark), and a wireless LAN. Further, when wireless communication is used, wired connection between the ultrasonic probe 10 and the external device 20 is not required. Thus, for example, the inconvenience, such as a cable getting in the way during the use of the ultrasonic probe 10, can be reduced.

Note that, in the present embodiment, description is made on an example in which the sensor communication unit 13 communicates with the external device 20 via wireless communication. Alternatively, wired communication may be used.

The housing 14 accommodates the ultrasonic sensor 11, the sensor circuit unit 12, and the sensor communication unit 13. The housing 14 is provided with a window portion 141 passing therethrough from the inside to the outside, and an ultrasonic wave transmission/reception surface of the ultrasonic sensor 11 is exposed from the window portion 141.

The ultrasonic probe 10 described above is brought into contact with the living body while the acoustic matching material such as an acoustic gel is interposed between the ultrasonic wave transmission/reception surface of the ultrasonic sensor 11 and the living body. The ultrasonic probe 10 may be brought into contact with a measurement part of the living body while being held by a user in the hand, or may be fixed to the living body by a fixing tool such as a band. In either case, the acoustic matching material is applied onto the transmission/reception surface of the ultrasonic sensor 11 or the measurement part of the living body for which the ultrasonic measurement is executed, and an operation is performed so as to prevent an air layer from being interposed between the ultrasonic sensor 11 and the living body.

### Configuration of External Device 20

The external device 20 is a device that is communicably connected to the ultrasonic probe 10. In the present embodiment, the external device 20 is provided independently from the ultrasonic probe 10, and is communicably connected to the ultrasonic probe 10 via wireless communication as described above.

The external device 20 is a device that controls the ultrasonic probe 10, and a general computer such as a smartphone, a tablet terminal, and a personal computer can be used.

Further, the external device 20 instructs the ultrasonic probe 10 to start the ultrasonic measurement, and acquires the ultrasonic signal transmitted from the ultrasonic probe 10 via wireless communication.

Specifically, as illustrated in FIG. 1, the external device 20 is configured to include a display unit 21, an input operation unit 22, a device storage 23 that stores various information, one or a plurality of processors 24, a device communication unit 25, and the like.

The display unit 21 displays various information under control of the processor 24. The display unit 21 may be configured freely as long as it is configured as a device that displays an image. Examples thereof include a liquid crystal display and an organic EL display.

The input operation unit 22 receives an input operation from a user. For example, the input operation unit 22 may be configured to include an operation button, an operation knob, or the like, may be an input device that is communicably connected to the external device 20, such as a keyboard and a mouse, or may be a touch panel configured integrally with the display unit 21.

The device storage 23 is a recording medium that stores various programs including a measurement program according to the ultrasonic measurement using the ultrasonic probe 10 and various types of data used in the various programs. Further, in the present embodiment, the device storage 23 is the storage of the present disclosure, and sequentially records and accumulates the ultrasonic signals that are transmitted from the ultrasonic probe 10 during the ultrasonic measurement.

The processor 24 executes various types of arithmetic processing by reading and executing the various programs stored in the device storage 23. Further, the processor 24 functions as a start request reception unit 241, a time display control unit 242, a signal acquisition unit 243, a signal arithmetic unit 244, and the like, by reading and executing the measurement program recorded in the device storage 23.

The start request reception unit 241 receives an input operation for starting the ultrasonic measurement, from the input operation unit 22. Further, when the input operation for starting the ultrasonic measurement is received from the input operation unit 22, the measurement start request for starting the ultrasonic measurement is output to the ultrasonic probe 10. In other words, in the present embodiment, the start request reception unit 241 functions as the instruction unit of the present disclosure. When the measurement start request is output, the time display control unit 242 causes the display unit 21 to display a time given before acquisition of the ultrasonic signal is started (hereinafter, referred to as a the stand-by time) and to count down the displayed time.

The signal acquisition unit 243 acquires the ultrasonic signal input from the ultrasonic probe 10, and records the ultrasonic signal in the device storage 23. More specifically, the signal acquisition unit 243 starts recording the ultrasonic signal that is input when the stand-by time being set in advance elapses after the start request reception unit 241 outputs the measurement start request.

In other words, in the present embodiment, when the start request reception unit 241 outputs the measurement start request to the ultrasonic probe 10, the sensor circuit unit 12 starts the ultrasonic wave transmission/reception processing (ultrasonic measurement), and the ultrasonic signal is transmitted from the ultrasonic probe 10 to the external device 20. However, recording of the ultrasonic signal is not started until the stand-by time elapses. The matter “the ultrasonic signal is not recorded” described herein indicates that recording of the ultrasonic signal as measurement data of the ultrasonic measurement is not executed. In the present embodiment, the signal acquisition unit 243 discards the ultrasonic signal that is input before the stand-by time elapses, and records the ultrasonic signal after the stand-by time elapses, as the measurement data, in the device storage 23.

Further, a predetermined fixed value may be used for the stand-by time, or the stand-by time may be changed as appropriate by a user. Alternatively, the stand-by time may be a time that is set by machine-learning an operation state of the ultrasonic probe 10 by a user.

The signal arithmetic unit 244 executes an arithmetic operation to acquire information relating to the living body, such as a thickness of a tissue (for example, a muscle, fat, and an organ), and a pulse rate, based on the ultrasonic signal recorded in the device storage 23.

The ultrasonic signal input from the ultrasonic probe 10 is raw data acquired by converting an analog signal to a digital signal, and includes the ultrasonic signal having a signal value corresponding to a sound pressure of the received ultrasonic wave and the reception timing of the ultrasonic wave (the elapse time from the ultrasonic wave transmission start timing). The signal arithmetic unit 244 can specify a plurality of depth degrees at which the ultrasonic waves are reflected, by analyzing those ultrasonic signals. Further, the signal arithmetic unit 244 can specify a boundary position of predetermined tissues (for example, a muscle and fat), based on the depth degrees, and can calculate a tissue thickness. Further, a blood vessel position is specified, and a change amount per unit time at the blood vessel position is detected. With this, a pulse rate can be measured.

### Ultrasonic Measurement Method

Next, an ultrasonic measurement method in the present embodiment is described.

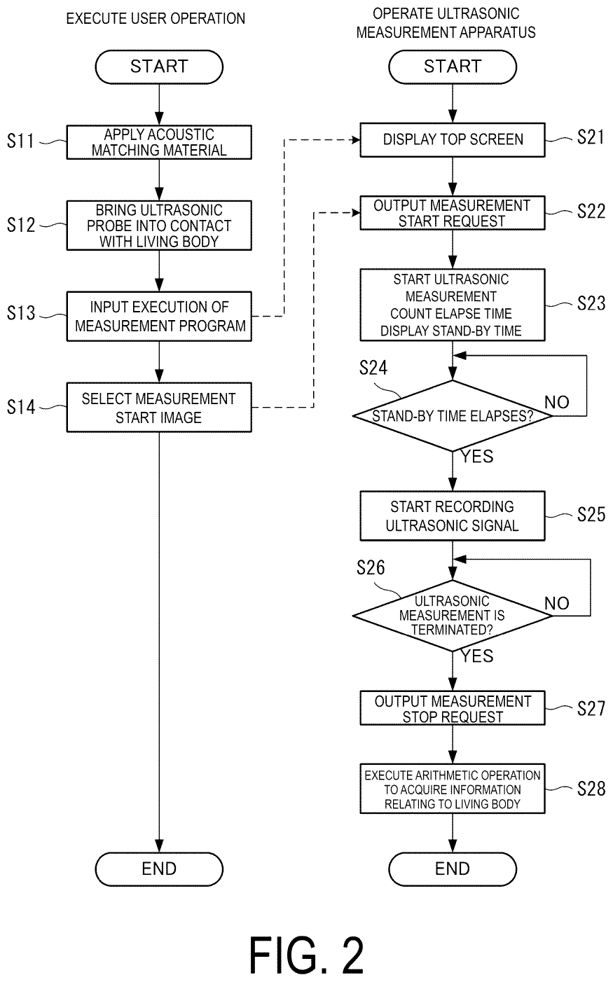

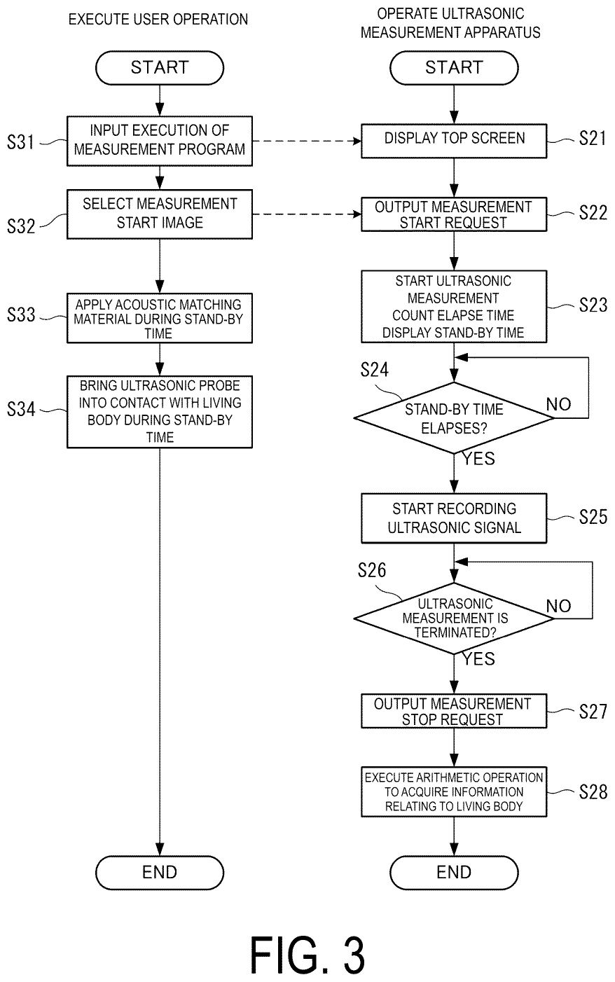

FIG. 2 and FIG. 3 are flowcharts illustrating the ultrasonic measurement method in the present embodiment.

When the ultrasonic measurement apparatus 1 is used to execute the ultrasonic measurement with respect to the living body, there are two pattens, as described above. In one case (pattern A), the input operation for starting the ultrasonic measurement is executed after a user brings the ultrasonic probe 10 into contact with the living body or fixes the ultrasonic probe 10 to the living body. In one case. In the other case (pattern B), a user executes the input operation for starting the ultrasonic measurement, and then brings the ultrasonic probe 10 into contact with the living body or fixes the ultrasonic probe 10 to the living body. FIG. 2 is a flowchart in the pattern A, and FIG. 3 is a flowchart in the pattern B.

In the pattern A, first, a user applies the acoustic matching material, such as an acoustic gel, onto the transmission/reception surface of the ultrasonic sensor 11 of the ultrasonic probe 10 or the measurement part of the living body (Step S11).

After that, a user brings the ultrasonic probe 10 into contacts with the measurement part of the living body and holds the ultrasonic probe 10 in that state, or fixes the ultrasonic probe 10 to the measurement part of the living body (Step S12).

After that, a user uses the external device 20 to input an instruction for executing the predetermined measurement program (Step S13).

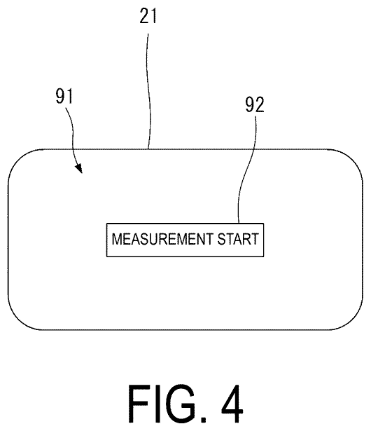

FIG. 4 is a diagram illustrating one example of an image displayed by the display unit 21 of the external device 20.

When the measurement program is executed, the start request reception unit 241 of the external device 20 displays a top screen 91 as illustrated in FIG. 4 (Step S21). For example, the top screen 91 includes a measurement start image 92 (for example, an icon image) for urging the start of the ultrasonic measurement.

A user operates the input operation unit 22 to select the measurement start image 92 (Step S14), and then the start request reception unit 241 outputs the measurement start request to the ultrasonic probe 10 (Step S22).

When the measurement start request is received via the sensor communication unit 13, the sensor circuit unit 12 of the ultrasonic probe 10 controls the ultrasonic sensor 11 to start the ultrasonic measurement. At the same time, the signal acquisition unit 243 counts the elapse time from the output of the measurement start request, and the time display control unit 242 controls the display unit 21 to display the stand-by time (Step S23).

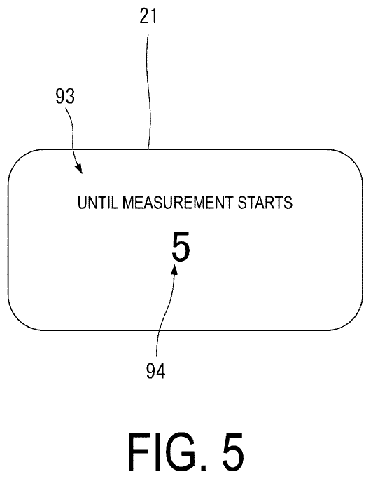

FIG. 5 is a diagram illustrating one example of a stand-by time image displayed by the display unit 21. As illustrated in FIG. 5, the time display control unit 242 causes the display unit 21 to display a stand-by time screen 93. On the stand-by time screen 93, a time image 94 indicating the stand-by time is displayed, and the stand-by time indicated in the time image 94 is reduced according to the elapse of time (countdown).

Further, the signal acquisition unit 243 determines whether the stand-by time elapses from the output of the measurement start request (Step S24). When it is determined as NO in Step S24, the stand-by time displayed by the display unit 21 is being counted down. During the stand-by time, although the ultrasonic signal is transmitted from the ultrasonic probe 10 to the external device 20 via the sensor communication unit 13, the signal acquisition unit 243 does not adopt the ultrasonic signal as the measurement data (does not record the ultrasonic signal in the device storage 23), and continues counting the stand-by time.

When it is determined as YES in Step S24, in other words, when the stand-by time elapses from the output of the measurement start request, and the stand-by time displayed by the display unit 21 is “0”, the signal acquisition unit 243 adopts, as the measurement data, the ultrasonic signal from the ultrasonic probe 10 via the sensor communication unit 13, and sequentially records the ultrasonic signal in the device storage 23 (Step S25).

The ultrasonic signal recorded as the measurement data in the device storage 23 is a signal from the ultrasonic measurement executed when the acoustic matching material is applied onto the transmission/reception surface of the ultrasonic probe 10 or the measurement part of the living body, and the ultrasonic probe 10 is brought into contact with or fixed to the measurement part of the living body. Therefore, in a case of the pattern A, the ultrasonic signal being a ultrasonic measurement result relating to the living body is appropriately recorded in the device storage 23.

After that, the signal acquisition unit 243 determines whether to terminate the ultrasonic measurement (Step S26). For example, when a user inputs a termination request for terminating the measurement, the signal acquisition unit 243 determines that the ultrasonic measurement is terminated. Alternatively, it may be set that the ultrasonic measurement is terminated after a predetermined measurement time being set in advance. In this case, the signal acquisition unit 243 is only required to determine whether the measurement time elapses from the start of recording of the ultrasonic signal in Step S25. When it is determined as NO in Step S26, the ultrasonic measurement continues, and recording of the ultrasonic signal in Step S25 continues.

When it is determined as YES in Step S26, the signal acquisition unit 243 outputs, to the ultrasonic probe 10, a measurement stop request for requesting the ultrasonic probe 10 to stop the ultrasonic measurement (Step S27). With this, the ultrasonic probe 10 stops driving of the ultrasonic sensor 11.

After that, the signal arithmetic unit 244 executes an arithmetic operation to acquire information relating to the living body (for example, an arithmetic operation for a tissue thickness, measurement of a pulse rate, and the like), based on the ultrasonic signals accumulated in the device storage 23 (Step S28).

Next, the pattern B is described.

In the pattern B, first, a user uses the external device 20 to input an instruction for executing the measurement program (Step S31).

With this, similarly to the pattern A, the external device 20 executes Step S21, the start request reception unit 241 causes the display unit 21 to display the top screen 91 for urging the start of the ultrasonic measurement.

A user operates the input operation unit 22 to select the measurement start image 92 for requesting the start of the ultrasonic measurement (Step S32). With this, similarly to Step S22, the start request reception unit 241 outputs the measurement start request to the ultrasonic probe 10. With this, similarly to Step S23, the ultrasonic sensor 11 starts the ultrasonic measurement, the signal acquisition unit 243 counts the elapse time from the output of the measurement start request, and the time display control unit 242 causes the display unit 21 to display the stand-by time screen 93 as illustrated in FIG. 5. Then, the countdown of the time in the time image 94 is started.

In the pattern B, the countdown of the stand-by time is displayed by the display unit 21. A user can recognize that the user is only required to complete contact between the ultrasonic probe 10 and the living body or fixing of the ultrasonic probe 10 to the living body by the end of the countdown, by visually recognizing the stand-by time.

Thus, during the stand-by time, while confirming the stand-by time displayed by the display unit 21, a user applies the acoustic matching material such as an acoustic gel onto the transmission/reception surface of the ultrasonic sensor 11 of the ultrasonic probe 10 or the measurement part of the living body (Step S33). Further, during the stand-by time, a user brings the ultrasonic probe 10 into contacts with the measurement part of the living body and holds the ultrasonic probe 10 in that state, or fixes the ultrasonic probe 10 to the measurement part of the living body (Step S34).

After that, the signal acquisition unit 243 executes Step S24 to determine whether the stand-by time elapses from the output of the measurement start request. When it is determined as NO in Step S24, the signal acquisition unit 243 does not adopt the ultrasonic signal transmitted from the ultrasonic probe 10, as the measurement data (does not record the ultrasonic signal in the device storage 23), and continues the count of the stand-by time. When it is determined as YES in Step S24, Step S25 is executed, and the signal acquisition unit 243 adopts the ultrasonic signal input from the ultrasonic probe 10, as the measurement data, and sequentially records the ultrasonic signal in the device storage 23.

In the pattern B, before the acoustic matching material is applied onto the ultrasonic sensor 11, or before the ultrasonic probe 10 is brought into contact with the living body, the ultrasonic measurement by the ultrasonic sensor 11 is started. However, during the stand-by time, the ultrasonic signal is not recorded in the device storage 23. Further, during the stand-by time, a user executes the operation in Step S33 and Step S34. With this, similarly to the pattern A, there can be acquired the ultrasonic signal from the ultrasonic measurement executed when the acoustic matching material is applied onto the transmission/reception surface of the ultrasonic probe 10 or the measurement part of the living body, and the ultrasonic probe 10 is brought into contact with or fixed to the measurement part of the living body. Therefore, in the present embodiment, even in a case of the pattern B, the ultrasonic signal being a ultrasonic measurement result relating to the living body is appropriately recorded in the device storage 23.

After that, Step S26 to Step S28 are executed. In other words, the signal acquisition unit 243 determines whether to terminate the ultrasonic measurement. When it is determined that the ultrasonic measurement is terminated, the measurement stop request is transmitted to the ultrasonic probe 10, and the ultrasonic measurement is terminated. Further, the signal arithmetic unit 244 executes an arithmetic operation to acquire information relating to the living body (for example, an arithmetic operation for a tissue thickness, measurement of a pulse rate, and the like), based on the ultrasonic signals accumulated in the device storage 23.

Herein, as described above, the signal arithmetic unit 244 executes an arithmetic operation to acquire information relating to the living body, a boundary position of target tissues is specified based on the ultrasonic signal. The boundary position may include an error when relying solely on data from a single transmission and a single reception of the ultrasonic wave. Thus, the boundary position is specified based on an average value of the measurement data from a plurality of transmissions and receptions of the ultrasonic wave. In this state, in a case in which the ultrasonic signal is taken in as the measurement data directly after the output of the measurement start request, when a user inputs the input operation for starting the ultrasonic measurement, and then brings the ultrasonic probe 10 into contact with the living body as in the pattern B, the ultrasonic signal acquired before the ultrasonic probe 10 is brought into contact with the living body is not data relating to the living body but an inadequate signal. Therefore, when such an inadequate ultrasonic signal is used as part of the measurement data to specify the boundary position of the tissues, a measurement error is increased. Although it is considered to use an algorithm for discarding, as unnecessary noise data, an ultrasonic signal having a signal value significantly different from those of other signals, such an algorithm is required to be introduced additionally. Further, when a threshold value for determining unnecessary data is not set appropriately, there may be a risk of discarding the measurement data that should not be discarded.

In contrast, in the present embodiment, when the predetermined stand-by time elapses after the input operation for starting the ultrasonic measurement is input, and the measurement start request is output, the ultrasonic signal is recorded in the device storage 23. Therefore, during the stand-by time, a user applies the acoustic matching material onto the ultrasonic wave transmission/reception surface of the ultrasonic probe 10 or the measurement part of the living body, and brings the ultrasonic probe 10 into contact with the living body. With this, acquisition of an erroneous ultrasonic signal can be suppressed. Therefore, the signal arithmetic unit 244 can execute an arithmetic operation to appropriately acquire information relating to the living body, based on the acquired ultrasonic signal. In this state, an algorithm for removing noise data is not required, and hence the cost for introducing the algorithm is not required.

Further, in the present embodiment, even when a user operates the ultrasonic measurement apparatus 1 in the procedure from Step S11 to Step S13 as in the pattern A, or a user operates the ultrasonic measurement apparatus 1 in the procedure from Step S31 to Step S34 as in the pattern B, the operation executed by the ultrasonic measurement apparatus 1 is common in the processing in Step S21 to Step S26. In other words, it is not required to change the operation of the ultrasonic measurement apparatus 1 between the case of the pattern A and the case of the pattern B. Further, as described above, the ultrasonic signal is acquired when the stand-by time elapses after the measurement start request is output. Thus, the ultrasonic signal stored in the device storage 23 does not include noise data, and the signal arithmetic unit 244 can execute an arithmetic operation to acquire information relating to the living body at higher accuracy.

### Actions and Effects of Present Embodiment

The ultrasonic measurement apparatus 1 of the present embodiment executes the ultrasonic measurement in which the ultrasonic wave is transmitted to the living body being a measurement target, and the ultrasonic wave reflected inside the living body is received. Further, the ultrasonic measurement apparatus 1 includes the ultrasonic sensor 11 and the processor 24 that functions as the start request reception unit 241 and the signal acquisition unit 243. The ultrasonic sensor 11 transmits and receives the ultrasonic wave, and outputs the ultrasonic signal by receiving the ultrasonic wave. The start request reception unit 241 outputs the measurement start request for instructing start of the ultrasonic measurement. The signal acquisition unit 243 records the ultrasonic signal in the device storage 23. Further, the signal acquisition unit 243 records, in the device storage 23, the ultrasonic signal when the predetermined time (stand-by time) elapses after the start request reception unit 241 outputs the measurement start request.

With this, in the present embodiment, the ultrasonic signal that is input before the stand-by time elapses is not adopted as the measurement data. Thus, even in a case in which a user inputs an operation for starting the ultrasonic measurement before bringing the ultrasonic probe 10 into contact with the living body, when the ultrasonic probe 10 is brought into contact with the living body during the stand-by time, inconvenient acquisition of the ultrasonic signal, which is not measurement data relating to the living body, as the measurement Thus, even when the signal arithmetic data can be prevented. unit 244 executes an arithmetic operation to acquire information relating to the living body, an arithmetic operation can be executed to acquire a highly accurate measurement result, based on the ultrasonic signal recorded in the device storage 23.

In the present embodiment, the external device 20 includes the display unit 21 that displays information. Further, the processor 24 includes a time display unit that functions as the time display control unit 242 and causes the display unit to display the time from the time at which the start request reception unit 241 outputs the measurement start request to the time at which the signal acquisition unit records the ultrasonic signal.

With this, a user can easily visually recognize the stand-by time until the ultrasonic signal is recorded as the measurement data in the device storage 23. Thus, while confirming the stand-by time, a user can perform a work operation of applying the acoustic matching material onto the ultrasonic sensor 11 and a work operation of bringing the ultrasonic sensor 11 into contact with the living body, and inconvenient acquisition of the ultrasonic signal before bringing the ultrasonic sensor 11 into contact with the living body can be prevented more.

The ultrasonic measurement apparatus 1 of the present embodiment includes the external device 20 that is communicably connected to the ultrasonic probe 10 including the ultrasonic sensor 11.

With this configuration, information relating to the living body, which is acquired based on the ultrasonic signal, can be processed by the external device 20, and the configuration of the ultrasonic probe 10 provided with the ultrasonic sensor 11 can be simplified.

In the ultrasonic measurement apparatus 1 of the present embodiment, the start request reception unit 241 is provided to the external device 20. In other words, the processor 24 of the external device 20 functions as the start request reception unit 241.

With this, the configuration of the ultrasonic probe 10 provided with the ultrasonic sensor 11 can be simplified. Further, the ultrasonic probe 10 may be held while being brought into contact with the living body, or the ultrasonic probe 10 may be fixed to the living body by using a band or the like. Thus, when an operation means is provided to the ultrasonic probe 10, an operation may be complex. In contrast, in the present embodiment, the external device 20 is operated to start the ultrasonic measurement easily.

In the ultrasonic measurement apparatus 1 of the present embodiment, the external device 20 is provided with the device storage 23, and the ultrasonic signal is recorded in the device storage 23.

When the ultrasonic probe 10 is provided with a storage, the configuration of the ultrasonic probe 10 is complex, and the cost of the ultrasonic probe 10 is increased. In contrast, when the device storage 23 provided to the external device 20 is used, the configuration of the ultrasonic probe 10 can be simplified. In particular, when a general computer such as a smartphone and a tablet terminal is used as the external device 20, a storage medium provided to the computer, such as a hard disk and a semiconductor memory, can be used.

### Second Embodiment

In the first embodiment described above, when the ultrasonic sensor 11 of the ultrasonic probe 10 executes the ultrasonic measurement, the ultrasonic signal is directly transmitted from the sensor communication unit 13 to the external device 20. In this case, the signal acquisition unit 243 of the external device 20 counts the elapse time from the measurement start request, and records the ultrasonic signal transmitted from the ultrasonic probe 10 after the stand-by time elapses. In contrast, the ultrasonic probe 10 may be provided with a buffer memory (sensor storage) that temporarily stores the ultrasonic signal, and the sensor circuit unit 12 may record the ultrasonic signal in the sensor storage when the stand-by time elapses after the measurement start request is received.

Note that, in the following description, the matters previously described are denoted with the same reference symbols, and the description therefor is omitted or simplified.

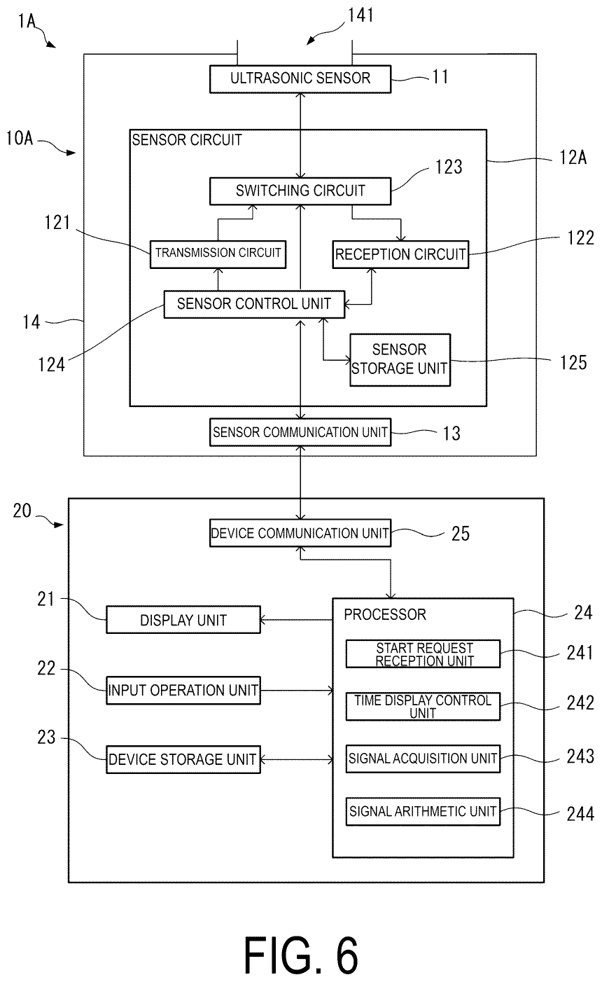

FIG. 6 is a block diagram illustrating an overview configuration of an ultrasonic measurement apparatus 1A of a second embodiment.

Similarly to the first embodiment, the ultrasonic measurement apparatus 1A of the present embodiment includes an ultrasonic probe 10A and the external device 20.

The ultrasonic probe 10A of the present embodiment includes the ultrasonic sensor 11, a sensor circuit unit 12A, the sensor communication unit 13, and the housing 14.

Further, the sensor circuit unit 12A of the present embodiment further includes a sensor storage 125 that temporarily stores the ultrasonic signal output from the ultrasonic sensor 11, in addition to the transmission circuit 121, the reception circuit 122, the switching circuit 123, and the sensor control unit 124.

Similarly to the first embodiment, the sensor control unit 124 of the present embodiment receives the measurement start request from the external device 20, and then controls the ultrasonic sensor 11 to execute the ultrasonic measurement. With this, the ultrasonic signal is input from the ultrasonic sensor 11 to the reception circuit 122, and the ultrasonic signal subjected to the signal processing by the reception circuit 122 is output.

Herein, In the present embodiment, the sensor control unit 124 temporarily stores the ultrasonic signal subjected to the signal processing, in the sensor storage 125, and transmits the ultrasonic signal to the external device 20 at the predetermined timing (for example, after the ultrasonic measurement processing is terminated). In this state, during the stand-by time from the measurement start request, the sensor control unit 124 discards the ultrasonic signal without recording the ultrasonic signal in the sensor storage 125. Further, the sensor control unit 124 records, in the sensor storage 125, the ultrasonic signal subjected to the signal processing by the reception circuit 122 after the stand-by time elapses.

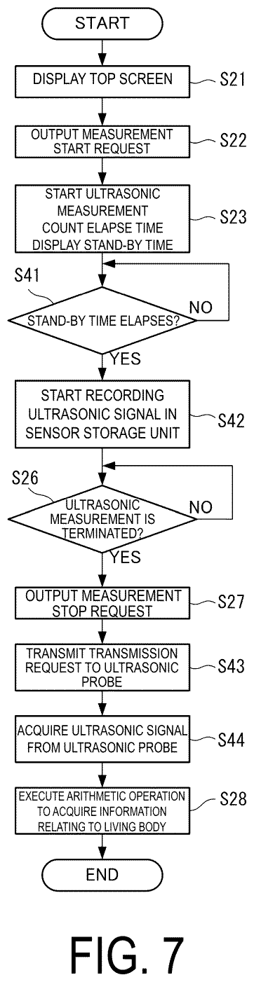

FIG. 7 is a flowchart illustrating an operation of the ultrasonic measurement apparatus 1A in the present embodiment. In the present embodiment, a user operates the external device 20 to input an instruction for executing the measurement program. With this, similarly to Step S21, the start request reception unit 241 of the external device 20 causes the display unit 21 to display the top screen 91 for urging the start of the ultrasonic measurement.

Further, a user operates the input operation unit 22 to select the measurement start image 92. With this, the start request reception unit 241 executes the processing in Step S22, and outputs the measurement start request to the ultrasonic probe 10. With this, similarly to Step S23, the ultrasonic sensor 11 starts the ultrasonic measurement. At the same time, the signal acquisition unit 243 counts the elapse time from the output of the measurement start request, and the time display control unit 242 causes the display unit 21 to display the stand-by time screen 93 as illustrated in FIG. 5. Then, the countdown of the time in the time image 94 is started.

Further, in the present embodiment, the sensor control unit 124 of the ultrasonic probe 10 executes the processing similar to Step S24, in other words, determines whether the stand-by time elapses after the measurement start request is received (Step S41).

When it is determined as NO in Step S41, the sensor control unit 124 discards the ultrasonic signal that is output from the ultrasonic sensor 11 via the reception circuit 122, and continues counting the stand-by time.

When it is determined as YES in Step S41, the sensor control unit 124 sequentially stores the ultrasonic signal, which is output from the ultrasonic sensor 11 via the reception circuit 122, in the sensor storage 125 (Step S42).

After that, the signal acquisition unit 243 executes Step S26 and Step S27. In other words, the signal acquisition unit 243 determines whether to terminate the ultrasonic measurement. When it is determined that the ultrasonic measurement is terminated, the measurement stop request is transmitted to the ultrasonic probe 10, and the ultrasonic measurement is terminated.

Further, after Step S27, the signal acquisition unit 243 transmits, to the ultrasonic probe 10, a transmission request of the ultrasonic signal (Step S43). With this, the sensor control unit 124 of the ultrasonic probe 10 transmits, to the external device 20, the ultrasonic signal recorded in the sensor storage 125, and the signal acquisition unit 243 acquires the ultrasonic signal from the ultrasonic probe 10 (Step S44). The acquired ultrasonic signal is recorded in the device storage 23.

After that, the signal arithmetic unit 244 executes Step S28, and executes an arithmetic operation to acquire information relating to the living body (for example, an arithmetic operation for a tissue thickness, measurement of a pulse rate, and the like), based on the ultrasonic signals recorded in the device storage 23.

The ultrasonic signal acquired in Step S44 is an ultrasonic signal acquired when the predetermined stand-by time after the measurement start request is output. Therefore, similarly to the first embodiment described above, even in a case in which a user uses the external device 20 to input an instruction for executing the measurement program before bringing the ultrasonic probe 10 into contact with the living body, as in the procedure of the pattern B, and then the measurement start request is output, when a user executes application of the acoustic matching material and an operation of bringing the ultrasonic probe 10 into contact with the living body during the stand-by time, an appropriate ultrasonic signal can be acquired.

### Actions and Effects of Present Embodiment

In the ultrasonic measurement apparatus 1A of the present embodiment, the ultrasonic probe 10A is provided with the sensor storage 125, and the ultrasonic signal is recorded when the stand-by time elapses after the measurement start request is output to the sensor storage 125.

With this, the actions and effects similar to those in the first embodiment can be achieved. Thus, even in a case in which a user inputs an operation for starting the ultrasonic measurement before bringing the ultrasonic probe 10 into contact with the living body, when the ultrasonic probe 10 is brought into contact with the living body during the stand-by time, inconvenient acquisition of the ultrasonic signal, which is not measurement data relating to the living body, as the measurement data can be prevented.

### Third Embodiment

In the first embodiment described above, description is made on an example in which a user uses the external device 20 to perform an operation for starting the ultrasonic measurement, and then the start request reception unit 241 outputs the measurement start request. However, the measurement start request is not limited to an input operation from a user.

In a third embodiment, description is made on an example in which the ultrasonic measurement apparatus automatically detects that the acoustic matching material is applied onto the ultrasonic wave transmission/reception surface of the ultrasonic sensor and the measurement start request is output.

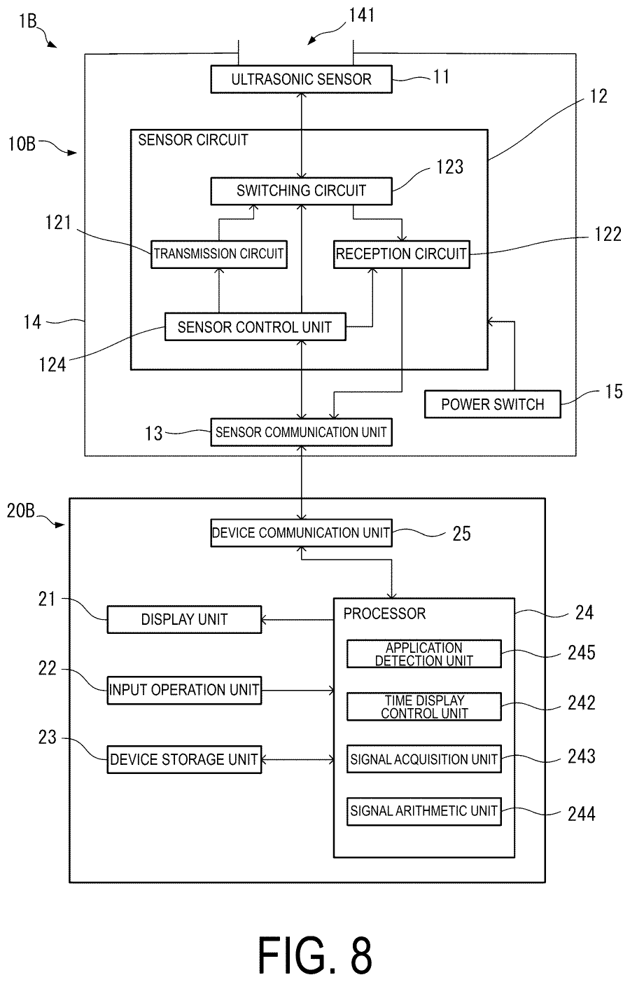

FIG. 8 is a block diagram illustrating an overview configuration of the ultrasonic measurement apparatus 1B of the third embodiment. In the present embodiment, the ultrasonic measurement apparatus 1B includes an ultrasonic probe 10B and an external device 20B.

The ultrasonic probe 10B includes the first embodiment, the ultrasonic sensor 11, the sensor circuit unit 12, the sensor communication unit 13, and the housing 14. Further, the ultrasonic probe 10B of the present embodiment includes a power switch 15 for driving the ultrasonic sensor 11. A user operates the power switch 15 to execute switching from an off state to an on state, the power is supplied from the battery provided in the housing 14 to the sensor circuit unit 12 and the sensor communication unit 13, and the ultrasonic sensor 11 starts the ultrasonic measurement. In other words, the power switch 15 is the switch of the present disclosure, and switches supply and non-supply of the power to the ultrasonic sensor 11.

Further, similarly to the first embodiment, the external device 20B of the present embodiment includes the display unit 21, the input operation unit 22, the device storage 23, the one or plurality of processors 24, and the like. Further, the processor 24 of the present embodiment reads and executes the various programs stored in the device storage 23. With this, the processor 24 functions as the time display control unit 242, the signal acquisition unit 243, the signal arithmetic unit 244, and an application detection unit 245.

In the present embodiment, the start request reception unit 241 is not provided, and the power switch 15 is operated in the ultrasonic probe 10B as described above. With this, the ultrasonic measurement is executed.

With this, the external device 20B receives the ultrasonic signal after timing at which the power switch 15 executes switching to an on state.

Further, the processor 24 of the present embodiment functions as the application detection unit 245, and the application detection unit 245 determines whether the acoustic matching material is applied, based on a change of the ultrasonic signal transmitted from the ultrasonic probe 10.

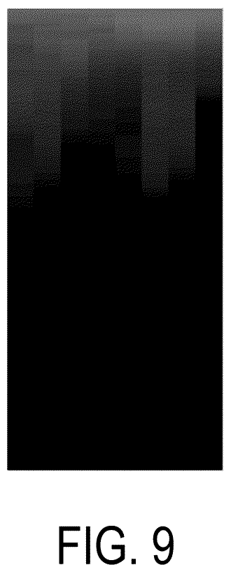

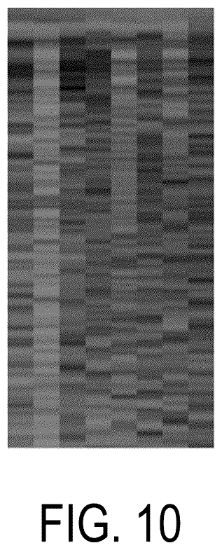

FIG. 9 is a diagram illustrating one example of a B-mode image generated based on the ultrasonic signal when the acoustic matching material is not applied onto the transmission/reception surface of the ultrasonic sensor 11. FIG. 10 is a diagram illustrating one example of the B-mode image generated based on the ultrasonic signal when the acoustic matching material is applied onto the transmission/reception surface of the ultrasonic sensor 11.

When the acoustic matching material is not applied onto the transmission/reception surface of the ultrasonic sensor 11, the ultrasonic wave reflected from the ultrasonic sensor 11 is not reflected, and hence the ultrasonic signal from the reflected ultrasonic wave is significantly reduced. Thus, when the B-mode image is generated based on the ultrasonic signal, an image in which a boundary line is not detected is acquired as illustrated in FIG. 9.

In contrast, when the acoustic matching material is applied onto the transmission/reception surface of the ultrasonic sensor 11, multiplexing reflections of the ultrasonic wave occur at a boundary between the acoustic matching material and an air layer and a boundary between the acoustic matching material and the transmission/reception surface. Therefore, the ultrasonic sensor 11 receives the ultrasonic wave from the multiplexing reflections. In other words, the ultrasonic signal having a great signal value is output from the ultrasonic sensor 11 at the cycle corresponding to the application thickness of the acoustic matching material. When the B-mode image is generated based on the ultrasonic signal from such multiplexing reflections, the image illustrated in FIG. 10 is acquired.

In this manner, the ultrasonic signal is changed before and after the acoustic matching material is applied onto the ultrasonic sensor 11. Therefore, the application detection unit 245 can determine that the acoustic matching material is applied onto the ultrasonic sensor 11 at timing at which the ultrasonic signal having an extremely small signal value is changed to the ultrasonic signal having a cyclic and intense signal value corresponding to multiplexing reflections.

Further, the application detection unit 245 outputs the measurement start request at timing at which the acoustic matching material is applied onto the ultrasonic sensor 11. In the present embodiment, in the ultrasonic probe 10, the power switch 15 is switched to an on state, and then the ultrasonic measurement by the ultrasonic sensor 11 is executed. Thus, the measurement start request is not necessarily transmitted to the ultrasonic probe 10.

For example, when the conditions of the ultrasonic wave for detecting the application of the acoustic matching material and the ultrasonic wave for the ultrasonic measurement with respect to the living body are the same, the application detection unit 245 is only required to output the measurement start request as a trigger for the count start of the stand-by time and the display of the time image 94.

However, when the frequency or the switching cycle of transmission and reception are changed between the ultrasonic wave for detecting the acoustic matching material and the ultrasonic wave for executing the ultrasonic measurement with respect to the living body, the measurement start request may be transmitted from the external device 20 to the ultrasonic probe 10. In this case, in the ultrasonic probe 10, when the power switch is switched from an off state to an on state, the sensor control unit 124 outputs, from the ultrasonic sensor 11, the ultrasonic wave in which the sound pressure, the frequency, and the switching cycle of transmission and reception of the ultrasonic wave for detecting the acoustic matching material are set. Further, when the measurement start request is input from the external device 20, the sensor control unit 124 outputs, from the ultrasonic sensor 11, the ultrasonic wave in which the sound pressure, the frequency, and the switching cycle of transmission and reception of the ultrasonic wave for the ultrasonic measurement with respect to the living body are set.

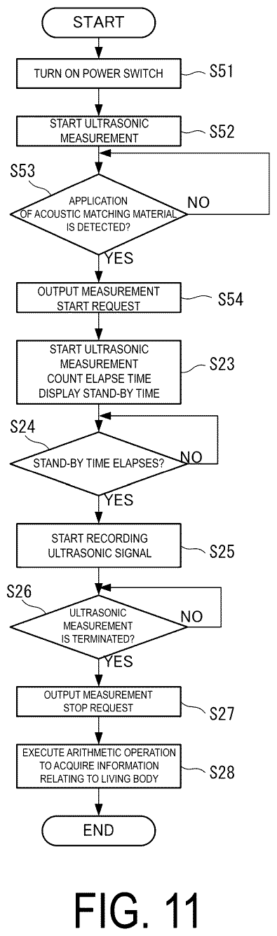

FIG. 11 is a flowchart illustrating the ultrasonic measurement method in the present embodiment.

In the present embodiment, a user operates the power switch 15 of the ultrasonic probe 10 to execute switching from an off state to an on state (Step S51).

With this, the sensor control unit 124 controls the ultrasonic sensor 11 to start the ultrasonic measurement (Step S52). With this, similarly to the first embodiment, the ultrasonic signal output from the ultrasonic sensor 11 is subjected to the signal processing by the reception circuit 122, and the ultrasonic signal subjected to the signal processing is transmitted from the sensor communication unit 13 to the external device 20B. Note that the ultrasonic wave transmitted to the external device 20B is not stored in the device storage 23.

Subsequently, the application detection unit 245 monitors the ultrasonic signal transmitted from the ultrasonic probe 10B in Step S52, and determines whether the acoustic matching material is applied onto the transmission/reception surface of the ultrasonic sensor 11, based on a change of the ultrasonic signal (Step S53).

When it is determined as NO in Step S53, in other words, when the ultrasonic signal having a great signal value is not received as the ultrasonic signal, based on the reception of the reflected wave, monitoring of the ultrasonic signal continues.

When, as the ultrasonic signal, the ultrasonic signal having a great signal value is acquired at the cycle corresponding to the thickness of the acoustic matching material, it is determined as YES by the application detection unit 245 in Step S53, in other words, it is determined that the acoustic matching material is applied. In this case, the application detection unit 245 outputs the measurement start request being a trigger for the count start of the stand-by time and the display of the time image 94 (Step S54). Note that, as described above, when the condition of the ultrasonic wave for detecting the acoustic matching material and the condition of the ultrasonic wave for the ultrasonic measurement with respect to the living body are different from each other, an operation may be executed so that the ultrasonic sensor 11 outputs the ultrasonic wave for the ultrasonic measurement with respect to the living body by outputting the measurement start request to the ultrasonic probe 10 receiving the measurement start request by the sensor control unit 124.

With this, similarly to Step S23, the ultrasonic sensor 11 continuously executes the ultrasonic measurement. At the same time, the signal acquisition unit 243 counts the elapse time from the output of the measurement start request, and the time display control unit 242 causes the display unit 21 to display the stand-by time screen 93 as illustrated in FIG. 5. Then, the countdown of the time in the time image 94 is started.

The processing thereafter is similar to that in the first embodiment, and the processing from Step S24 to Step S28 is executed.

### Actions and Effects of Present Embodiment

In the ultrasonic measurement apparatus 1B of the present embodiment, the ultrasonic probe 10B is provided with the power switch 15 that switches supply and non-supply of the power to the ultrasonic sensor 11. When the power switch 15 is switched from an off state to an on state, the power is supplied, and the ultrasonic sensor 11 starts the ultrasonic wave transmission/reception processing (ultrasonic measurement). Further, the processor 24 of the external device 20B also functions as the application detection unit 245. The application detection unit 245 functions as the instruction unit of the present disclosure, and outputs the measurement start request, based on a change of the ultrasonic signal output from the ultrasonic sensor 11.

With this, it can be determined whether the acoustic matching material is applied onto the ultrasonic sensor 11, based on the ultrasonic signal, and the measurement start request can be output with the application of the acoustic matching material as a trigger. Thus, a user is not required to execute the input operation for starting the ultrasonic measurement, and a work operation of a user can be reduced.

### MODIFICATION EXAMPLES

Note that the present disclosure is not limited to each of the embodiments described above. The configurations achieved by combining variations, modifications, and each of the embodiments with each other within the scope in which the object of the present disclosure can be achieved are included in the present disclosure.

### First Modification Example

Similarly to the first embodiment, in the third embodiment, when the ultrasonic sensor 11 executes the ultrasonic measurement, the ultrasonic signal is transmitted from the ultrasonic probe 10B to the external device 20B, and the application detection unit 245 of the external device 20B detects the application of the acoustic matching material, based on the ultrasonic signal being received.

In contrast, as in the second embodiment, when the ultrasonic probe is provided with the sensor storage 125, and the ultrasonic signal is transmitted after the termination of the ultrasonic measurement, the sensor control unit 124 of the ultrasonic probe may function as the application detection unit.

In this case, the sensor control unit 124 determines whether the acoustic matching material is applied onto the transmission/reception surface of the ultrasonic sensor 11, based on the ultrasonic signal. Further, when it is determined that the acoustic matching material is applied, the sensor control unit 124 transmits the measurement start request to the external device.

When the measurement start request is received, the external device executes the processing in Step S23, the signal acquisition unit 243 counts the elapse time from the output of the measurement start request, and the time display control unit 242 causes the display unit 21 to display the stand-by time screen 93 as illustrated in FIG. 5. Then, the countdown of the time in the time image 94 is started.

Further, the sensor control unit 124 executes the processing in Step S41 to Step S42 in the second embodiment, starts counting the stand-by time when the measurement start request is output, and starts recording the ultrasonic signal when the stand-by time elapses. Hereinafter, the processing similar to that in the second embodiment is executed.

### Second Modification Example

In the embodiments described above, as an example, the ultrasonic probes 10, 10A, and 10B are connected to the external devices 20 and 20B via wireless communication, but may be configured to be connected thereto in a wired manner.

### Third Modification Example

In the first embodiment described above, the ultrasonic probe 10 and the external device 20 are provided independently from each other, the external device 20 executed the measurement program, and a user executes the input operation for starting the ultrasonic measurement. In this case, the start request reception unit 241 outputs the measurement start request to the ultrasonic probe 10. In contrast, as in the third embodiment, the ultrasonic probe 10 is configured to include the power switch 15, and the power switch 15 is switched from an off state to an on state. With this, the sensor control unit 124 may output the measurement start request.

In this case, when the measurement start request is received, the external device 20 may execute the processing in Step S23, the signal acquisition unit 243 may count the elapse time from the output of the measurement start request, and the time display control unit 242 may cause the display unit 21 to display the stand-by time screen 93 as illustrated in FIG. 5. Then, the countdown of the time in the time image 94 may be started. The processing thereafter is similar to that in the first embodiment.

Note that the same applies to the second embodiment. The ultrasonic probe 10A of the second embodiment is configured to include the power switch 15 as described in the third embodiment, and the power switch 15 is switched from an off state to an on state. In this manner, the sensor control unit 124 may output the measurement start request to the external device 20. In this case, the power switch 15 functions as the instruction unit of the present disclosure.

In this case, the sensor control unit 124 starts the count of the stand-by time with the output of the measurement start request as a trigger. Further, similarly, when the measurement start request is received, the external device executes the processing in Step S23, the signal acquisition unit 243 counts the elapse time from the output of the measurement start request, and the time display control unit 242 causes the display unit 21 to display the stand-by time screen 93 as illustrated in FIG. 5. Then, the countdown of the time in the time image 94 is started.

The processing thereafter is similar to that in the second embodiment.

### Fourth Modification Example

Further, the ultrasonic probe may be configured to include the function of the external device 20. In other words, the ultrasonic probe 10A of the second embodiment may be configured to include a display unit, the sensor control unit 124 may be configured to function as the start request reception unit 241, the time display control unit 242, the signal acquisition unit 243, the signal arithmetic unit 244, and the like. In this case, the sensor control unit 124 may be configured to further function as the application detection unit 245.

### Fifth Modification Example

In the embodiments described above, as an example, the external device 20, 20B is provided with the display unit 21, the time image 94 indicating the stand-by time is displayed by the display unit 21, and the stand-by time is counted down. However, a configuration in which the display unit 21 is not provided may be adopted.

For example, in place of the display unit 21, an audio output unit may be provided to announce the countdown of the stand-by time via audio.

Alternatively, the display unit 21 may not be provided with an audio output unit, and the stand-by time may not be announced. In this case, as long as a user recognizes that the stand-by time is set in advance, the user can execute a work operation to bring the ultrasonic probe 10 into contact with the living body during the stand-by time.

### Sixth Modification Example

In the embodiment described above, description is made on the matter that the time set in advance is used as the stand-by time or a user may set the stand-by time as appropriate. Moreover, the stand-by time may be automatically set for each user.

For example, in the first embodiment, when the measurement start request is output, the ultrasonic sensor 11 starts the ultrasonic measurement, and the ultrasonic signal is transmitted to the external device 20. The first embodiment is an example in which the ultrasonic signal is not adopted as the measurement data, and is discarded. The ultrasonic signal may be recorded as teaching data, not as the measurement data, in the device storage 23. For example, when the measurement start request is output before the acoustic matching material is applied onto the ultrasonic sensor 11 or the ultrasonic probe 10 is brought into contact with the living body, the ultrasonic signal is changed at the timing at which the acoustic matching material is applied onto the ultrasonic sensor 11 and the timing at which the ultrasonic sensor 11 is brought into contact with the living body. By detection such timing, the time from the output of the measurement start request to the application of the acoustic matching material onto the ultrasonic sensor 11 and the time until the ultrasonic sensor 11 is brought into contact with the living body can be calculated. Therefore, the processor 24 records, in the device storage 23, the time from the output of the measurement start request to the contact of the ultrasonic sensor 11 with the living body, and the time acquired by adding a predetermined marginal time to the average value or the maximum value may be set as the stand-by time. In this case, even when the work operation time relating to the application of the acoustic matching material or the work operation time for mounting the ultrasonic probe 10 on the living body differ for each user, personalization for each user using the ultrasonic measurement apparatus 1 can be achieved.

### Seventh Modification Example

In the embodiments described above, the living body is exemplified as the measurement target, but the measurement target is not limited thereto.

For example, the measurement target may be a structure such as a concrete article and a pipe. In particular, the present disclosure can be applied advantageously to a case in which the ultrasonic measurement is executed continuously for the predetermined time, and an arithmetic operation is executed to acquire the measurement result, based on the average of the ultrasonic signals acquired at the predetermined sampling cycle.

### Overview of Present Disclosure

According to a first aspect of the present disclosure, an ultrasonic measurement apparatus is an ultrasonic measurement apparatus configured to transmit an ultrasonic wave to a measurement target and receive the ultrasonic wave reflected inside the measurement target, and includes an ultrasonic sensor configured to transmit and receive the ultrasonic wave and output an ultrasonic signal by receiving the ultrasonic wave, an instruction unit configured to output a measurement start request for instructing start of the ultrasonic measurement, and a signal acquisition unit configured to record the ultrasonic signal in a storage, wherein the signal acquisition unit records, in the storage, the ultrasonic signal after a predetermined time elapses from the output of the measurement start request from the instruction unit.

With this, a user can bring the ultrasonic sensor into contact with the measurement target before the predetermined time elapses, and inconvenient recording of the ultrasonic signal, which is acquired when the ultrasonic sensor is not brought into contact with the measurement target, in the storage can be prevented. Therefore, an arithmetic operation can be executed to acquire a highly accurate measurement result, based on the ultrasonic signal recorded in the storage.

The ultrasonic measurement apparatus according to the present aspect may include a display unit configured to display information, and a time display control unit configured to cause the display unit to display a time from a time at which the instruction unit outputs the measurement start request to a time at which the signal acquisition unit starts recording the ultrasonic signal.

With this, while confirming the display unit, a user can easily visually recognize the time until the ultrasonic signal is recorded as the measurement data in the storage. Thus, while confirming the time, a work operation of applying the acoustic matching material onto the ultrasonic sensor and a work operation of bringing the ultrasonic sensor into contact with the living body can be performed.

The ultrasonic measurement apparatus according to the present aspect may include an external device being communicably connected to the ultrasonic sensor.

With this, the configuration of the ultrasonic probe can be simplified. In other words, a general computer such as a smartphone and a tablet terminal can be used as the external device. When an application for controlling the ultrasonic sensor is installed, a high-performance arithmetic circuit that executes a program for processing the measurement data is not required in the ultrasonic sensor. Thus, the configuration can be simplified.

In the ultrasonic measurement apparatus according to the aspect described above, the instruction unit may be provided to the external device.

The ultrasonic sensor may be maintained in contact with the measurement target, or may be fixed to the measurement target. Therefore, when an operation unit that instructs the start of the ultrasonic measurement is provided on the ultrasonic sensor side, the ultrasonic measurement may be complicated. In contrast, the external device is configured to include the instruction unit. With this, a user can operate the external device to easily execute the operation for starting the ultrasonic measurement.

In the ultrasonic measurement apparatus according to the aspect described above, the storage may be provided to the external device.

When the storage is provided to the ultrasonic sensor, the cost of the ultrasonic sensor is increased. In contrast, the external device is provided with the storage, and hence the cost of the ultrasonic sensor can be reduced. In particular, when a general computer such as a smartphone and a tablet terminal is used as the external device, a storage medium provided to the computer, such as a hard disk and a semiconductor memory, can be used.

In the ultrasonic measurement apparatus according to the present aspect, the storage may be provided to the ultrasonic sensor.

In this case, when the measurement start request is output, the ultrasonic wave after the predetermined elapses is recorded in the storage provided to the ultrasonic sensor. When the ultrasonic signal is output to the external device, the ultrasonic signal recorded in the storage provided to the ultrasonic sensor may be transmitted.

The ultrasonic measurement apparatus according to the present aspect may include a switch configured to switch supply and non-supply of power to the ultrasonic sensor, wherein the ultrasonic sensor starts transmission and reception of the ultrasonic wave when the switch supplies the power, and the instruction unit outputs the measurement start request, based on a change of the ultrasonic signal being output from the ultrasonic sensor.

With this, it can be determined whether the acoustic matching material is applied onto the ultrasonic sensor, based on the ultrasonic signal, and the measurement start request can be output with the application of the acoustic matching material as a trigger. In this case, a user is not required to execute the input operation for starting the ultrasonic measurement, and a work operation of a user can be reduced.

What is claimed is:

1. An ultrasonic measurement apparatus configured to transmit an ultrasonic wave to a measurement target and receive the ultrasonic wave reflected inside the measurement target, the ultrasonic measurement apparatus comprising:
an ultrasonic sensor configured to transmit and receive the ultrasonic wave and output an ultrasonic signal by receiving the ultrasonic wave;
an processor configured to output a measurement start request for instructing start of the ultrasonic measurement and record the ultrasonic signal in a storage, wherein
the processor records, in the storage, the ultrasonic signal after a predetermined time elapses from the output of the measurement start request.

2. An ultrasonic measurement apparatus according to claim 1, comprising:
a display unit configured to display information; and
a processor configured to cause the display unit to display a time from a time at which the processor outputs the measurement start request to a time at which the processor starts recording the ultrasonic signal.

3. An ultrasonic measurement apparatus according to claim 1, comprising:
an external device being communicably connected to the ultrasonic sensor.

4. An ultrasonic measurement apparatus according to claim 3, wherein
the processor is provided to the external device.

5. An ultrasonic measurement apparatus according to claim 3, wherein
the storage is provided to the external device.

6. An ultrasonic measurement apparatus according to claim 1, wherein
the storage is provided to the ultrasonic sensor.

7. An ultrasonic measurement apparatus according to claim 1, comprising:
a switch configured to switch supply of power and non-supply of power to the ultrasonic sensor, wherein
the ultrasonic sensor starts transmission of the ultrasonic wave and reception of the ultrasonic wave when the switch supplies the power, and
the processor outputs the measurement start request, based on a change of the ultrasonic signal being output from the ultrasonic sensor.
